(19) United States
(12) Patent Application Publication
(10) Pub. No.: US 2025/0244312 A1
(43) Pub. Date: Jul. 31, 2025

(54) EX VIVO COLONIC BIOPSY PLATFORM TO EVALUATE MULTI-OMIC SIGNATURES FOR SCREENING CANDIDATE THERAPEUTICS

(71) Applicant: Athos Therapeutics, Inc., Torrance, CA (US)

(72) Inventors: Dimitrios ILIOPOULOS, Los Angeles, CA (US); Iordanis KARAGIANNIDIS, North Hollywood, CA (US)

(21) Appl. No.: 18/040,422

(22) PCT Filed: Aug. 5, 2021
(86) PCT No.: PCT/US2021/044714

§ 371 Date: Feb. 2, 2023

Related U.S. Application Data
(60) Provisional application No. 63/062,954, filed on Aug. 7, 2020.

(51) Int. Cl. G01N 33/50; A61K 45/06; C12N 5/00; C12N 5/071; G01N 33/53; G01N 33/573; G01N 33/68

(52) U.S. Cl. CPC G01N 33/5044; A61K 45/06; C12N 5/0018; C12N 5/0679; G01N 33/5023; G01N 33/5308; G01N 33/573; G01N 33/6869; C12N 2501/998; C12N 2503/02; G01N 2333/525; G01N 2333/545; G01N 2333/91017; G01N 2500/10

(57) ABSTRACT

Some embodiments described herein relate to a method of screening candidate therapeutics for gastrointestinal diseases, including inflammatory bowel diseases, ulcerative colitis, and Crohn's Disease, using an ex vivo biopsy high throughput platform. Some embodiments relate to a high-throughput method of screening an ex vivo biopsy for chromatin modifications associated with an gastrointestinal disease. Some embodiments relate to a multi-omic method of screening candidate therapeutics for treatment of gastrointestinal diseases, including inflammatory bowel diseases, ulcerative colitis, and Crohn's Disease.

### RELATED APPLICATIONS

This application is the United States National Phase entry, under 35 U.S.C. § 371 of International Patent Application No. PCT/US2021/044714, filed Aug. 5, 2021, which claims priority to U.S. Provisional Patent Application No. 63/062,954, filed Aug. 7, 2020, the entire contents of each of which is incorporated by reference herein.

### BACKGROUND

Inflammatory Bowel Diseases (IBD) are chronic inflammatory diseases of the gastrointestinal tract that primarily consist of Ulcerative colitis (UC) and Crohn's Disease (CD). UC typically causes long-lasting inflammation and sores (ulcers) in the innermost lining of large intestine (colon) and rectum. CD is typically characterized by inflammation of the lining of digestive tract, which often spreads deep into affected tissues.

IBD can be debilitating and sometimes leads to life-threatening complications. The exact cause, or causes, of IBD remains unknown.

### INCORPORATION BY REFERENCE OF MATERIAL IN ASCII TEXT FILE

This application incorporates by reference herein the Sequence Listing contained in the following ASCII text file being concurrently submitted herewith: File name: ATHO.005NP_ST25.txt; created Nov. 30, 2023, which is 2,162 bytes in size.

### SUMMARY

Provided for herein, in several embodiments, are methods of screening candidate therapeutics and methods and uses for same in the treatment of gastrointestinal diseases, such as inflammatory bowel diseases, including ulcerative colitis and Crohn's Disease, using an ex vivo biopsy high throughput platform.

According to several embodiments, there is provided an ex vivo method of screening one or more candidate therapeutics for treatment of a gastrointestinal disease, the method comprising receiving a biopsy sample from a patient with at least one active disease site, separating the biopsy sample into multiple replicates and contacting one or more of the replicates with an artificial tissue culture medium, with at least a portion of the replicates being exposed to a candidate therapeutic and one or more of the replicates is maintained as a control (e.g., exposure to the artificial tissue culture medium but not a candidate therapeutic). In several embodiments, the replicates (whether experimental or control) are cultured for a period of time and, after the period of time, the method comprises measuring levels of one or more biomarkers from a replicate cultured with a candidate therapeutic and from a replicate cultured without a candidate therapeutic, wherein the one or more biomarkers comprise at least one biomarker indicative of a histone modification, and wherein a candidate therapeutic is identified when the candidate therapeutic induced changes in the at least one biomarker indicative of a histone modification such that the measured biomarker from the replicate cultured with the candidate therapeutic is significantly different from the measured biomarker from the replicate cultured without the candidate therapeutic.

In several embodiments, the gastrointestinal disease is selected from inflammatory bowel disease, celiac disease, irritable bowel syndrome, and esophagitis. In several embodiments, the at least one biomarker indicative of a histone modification is measured by ELISA, Western Blot, mass spectroscopy, immunohistochemistry or other technique to detect protein expression.

In several embodiments, the at least one biomarker indicative of a histone modification measured is H3K9me2 or H3K27me3. In additional embodiments, both H3K9me2 and H3K27me3 are measured. In several embodiments, a panel of biomarkers indicative of a histone modification are measure, such as two, three, four, or more of H3K9me1/2/3, H3K27me1/2/3, H3K4me1/2/3, H3K36me1/2/3, H3K79me1/2/3, H3K9ac, H3K14ac, H3K 18acm H3K56ac, H3ser 10P, H3ser28P, total H3, H4K5ac, H4K8ac, H4K12ac, H4K16ac, H4R3m2a, H4R3m2s, H4K20me1/2/3, H4ser1, total H4, H3S10ph and H3S28ph, and/or the related histone-modifying genes: ASHIL, DOTIL, EHMT1, G9A/EHMT2,EZH1, EZH2, BMI1, SUZ12, MLL1-5, NSD1, PRDM2, SETD1-9, SETDB1/2, SETMAR, SMYD1-5, SUV39H1/2, SUV420H1/2, PRMT1-7, KDM1A/B, KMD2A/B, KMD3A/B, JMJD1C, KMD4A/B/C/D, KDM5A/B/C/D, KDM6A/B, GCN5, PCAF, HBO1,p300, CBP, SRC-1, ACTR, TIF2, TAF1, TFIIIC, HDAC1-11, and/or SIRT1-7.

In several embodiments, the method further comprises measuring the level of at least one cytokine associated with a gastrointestinal disease. In several embodiments, the at least one cytokine level measured is tumor necrosis factor alpha (TNFA) or interleukin 1B (IL1B). In several embodiments, both TNFA and IL1B are measured. In several embodiments, a panel of cytokines are measured, such as two, three, four, or more of TNFA, ILIB, ILIRA, IL2, IL4, IL5, IL6, IL7, IL8, IL10, IL12, IL13, IL15, IL17A, IL17E, IL33, GM-CSF, G-CSF, ICAM1, MCP-1, IFNgamma. MIP1A, MIP1B, CCL5/RANTES, CCL11, CD40, CX3CL1, CXCL1, CXCL2, CXCL10, TREM1, MIP3A, and/or MIP3B. In several embodiments, the at least one cytokine level is measured by ELISA, Western Blot, mass spectroscopy, immunohistochemistry or other technique to detect protein expression.

In one embodiment, TNFA, IL1B, H3K9me2, and H3K27me3 are all measured by ELISA.

In several embodiments, the artificial tissue culture medium used to contact the replicates comprises L-alanyl-L-glutamine dipeptide. In several embodiments, the concentration of L-alanyl-L-glutamine dipeptide in the artificial tissue culture medium is between about 1 mM and about 20 mM. In several embodiments, the concentration of L-alanyl-L-glutamine dipeptide is between about 2 mM and about 6 mM. In one embodiment, the concentration of L-alanyl-L-glutamine dipeptide is about 4 mM. In several embodiments, the contacting of the replicates with artificial tissue culture medium occurs in a tissue culture plate, well, or related culture receptacle.

In several embodiments, other biomarkers are evaluated. For example, in several embodiments, the method further comprises measuring the expression levels of one or more mature microRNAs. In some embodiments, the method further comprises measuring the expression levels of at least one epitranscriptomic regulator selected from the group consisting of a methyltransferase, a demethylase, a reader, a deaminase, and combinations thereof. In several embodiments, the methyltransferase is selected from METTL3, METTL14, METTL1, METTL6, METTL13, METTL16, NSUN2, NSUN3, NSUN4, TRMT2A, TRMT6, TRMT11, TRMT12, TRMT16B, BCDIN3D, KIAA1456, HENMT1, FBL, DIMT1, and combinations thereof. In several embodiments, the demethylase is selected from ALKBH3, ALKBH5, FTO and combinations thereof. In several embodiments, the reader is one or both of YTHDC2 and YTHDF2. In several embodiments, the deaminase is one or both of ADAR1, ADAR2.

In several embodiments, the biopsy sample is obtained by colonoscopy, sigmoidoscopy, surgery, endoscopy, or gastroscopy. In several embodiments, a gastrointestinal tissue same is obtained during, for example, a surgical procedure in which the gastrointestinal tract is undergoing an operation or is otherwise accessible. In several embodiments, the biopsy sample is from the epithelial mucosa of a patient. In other embodiments, the biopsy sample is from the esophagus, stomach, duodenum, small or large intestine of a patient. In several embodiments, the biopsy sample is from a patient who is newly diagnosed with Ulcerative colitis, Crohn's Disease, Irritable Bowel Syndrome, Esophagitis, Celiac Disease, or has been treated with steroids, immunomodulators, and/or biologics. In several embodiments, the biopsy sample is from a patient with an active disease site. In several embodiments, the biopsy sample is separated into from 2 to 100 replicates so as to allow a high-throughput screening of candidate therapeutics.

In several embodiments, there is also provided a method of treating a patient having an gastrointestinal disease comprising administering a therapeutically effective amount of a candidate therapeutic identified by the screening methods disclosed herein.

In several embodiments, there is provided a method for identifying an effective compound for treatment of or use in treating an gastrointestinal disease, the method comprising separating a biopsy sample from a subject believed to have or having a gastrointestinal disease into a plurality of replicates, placing a plurality of the replicates in a tissue culturing environment comprising an artificial tissue culture medium, adding, to one or more of the tissue culturing environments, a candidate therapeutic, wherein at least one of the plurality of replicates is maintained as a control and is placed in an tissue culturing environment comprising an artificial tissue culture medium but not a candidate therapeutic, culturing the plurality of placed replicates for a period of time; and measuring, after the period of time, levels of one or more biomarkers from a replicate cultured with a candidate therapeutic and from a replicate cultured without a candidate therapeutic, wherein a candidate therapeutic is identified when the candidate therapeutic induced changes in the one or more biomarkers such that the measured one or more biomarkers from the replicate cultured with the candidate therapeutic is significantly different from the measured one or more biomarkers from the replicate cultured without the candidate therapeutic.

A candidate therapeutic for the treatment of a gastrointestinal disease, wherein the candidate therapeutic was selected for use in the treatment by a method comprising culturing a plurality of replicates of a biopsy sample from a patient with at least one active gastrointestinal disease in an artificial tissue culture medium, wherein each replicate is placed in a tissue culturing environment, adding, to one or more of the tissue culturing environments, a candidate therapeutic, wherein at least one of the plurality of replicates is maintained as a control and is placed in an individual tissue culturing environment comprising an artificial tissue culture medium but not a candidate therapeutic, culturing the plurality of placed replicates for a period of time, and measuring, after the period of time, levels of one or more biomarkers from a replicate cultured with a candidate therapeutic and from a replicate cultured without a candidate therapeutic, wherein a candidate therapeutic is identified when the candidate therapeutic induced changes in the one or more biomarkers such that the measured one or more biomarkers from the replicate cultured with the candidate therapeutic is significantly different from the measured one or more biomarkers from the replicate cultured without the candidate therapeutic.

In several embodiments, the candidate therapeutic comprises, consists of, or consists essentially of, one or a combination of: a histone modifying factor selected from the group consisting of ASH1L, DOT1L, EHMT1, G9A/EHMT2,EZH1, EZH2, BMI1, SUZ12, MLL1-5, NSD1, PRDM2, SETD1-9, SETDB1/2, SETMAR, SMYD1-5, SUV39H1/2, SUV420H1/2, PRMT1-7, KDM1A/B, KMD2A/B, KMD3A/B, JMJDIC, KMD4A/B/C/D, KDM5A/B/C/D, KDM6A/B, GCN5, PCAF, HBO1,p300, CBP, SRC-1, ACTR, TIF2, TAF1, TFIIIC, HDAC1-11, SIRT1-7, and combinations thereof, a chemical compound targeting a cytokine or chemokine selected from the group consisting of TNFA, IL1B, IL1RA, IL2, IL4, IL5, IL6, IL7, IL8, IL10, IL12, IL13, IL15, IL17A, IL17E, IL33, GM-CSF, G-CSF, ICAM1, MCP-1, IFNgamma. MIPIA, MIP1B, CCL5/RANTES, CCL11, CD40, CX3CL1, CXCL1, CXCL2, CXCL10, TREM1, MIP3A, MIP3B, and combinations thereof, a chemical compound targeting an epitranscriptomic regulator, wherein the epitranscriptomic regulator is a methyltransferase, wherein the methyltransferase is selected from the group consisting of METTL3, METTL14, METTL1, METTL6, METTL13, METTL16, NSUN2, NSUN3, NSUN4, TRMT2A, TRMT6, TRMT11, TRMT12, TRMT16B, BCDIN3D, KIAA1456, HENMT1, FBL, DIMT1, and combinations thereof a chemical compound targeting an epitranscriptomic regulator, wherein the epitranscriptomic regulator is a demethylase, wherein the demethylase is selected from the group consisting of ALKBH3, ALKBH5, FTO, and combinations thereof,

a chemical compound targeting an epitranscriptomic regulator, wherein the epitranscriptomic regulator is a reader, wherein the reader comprises one or both of YTHDC2 and YTHDF2, a chemical compound targeting an epitranscriptomic regulator, wherein the epitranscriptomic regulator is a deaminase, wherein the deaminase comprises one or both of ADAR1 and ADAR2, and/oran miR-24-3p inhibitor, wherein the miR-24-3p inhibitor is an antisense-miR-24-3p compound, and/or wherein the antisense-miR-24-3p compound has the sequence of SEQ ID NO: 1 (5-CTGmCTGAAmCTGAGmCCA-3) or SEQ ID NO: 2 (5-CTGCTGAACTGAGCCA-3).

Also provided for herein is the use of a candidate therapeutic as disclosed herein for the treatment of a gastrointestinal disease and/or in the manufacture of a medicament for the treatment of a gastrointestinal disease.

Provided for herein are methods of treatment of a gastrointestinal disease comprising administering to the subject a therapeutically effective amount of a candidate therapeutic provided for herein.

Provided for herein in several embodiments is a high-throughput method of screening an ex vivo intestinal biopsy for chromatin modifications associated with a gastrointestinal disease, comprising separating an intestinal biopsy sample from a patient with at least one active gastrointestinal disease into a plurality of replicates, culturing the replicates in an artificial medium with each replicate in a tissue culturing environment, wherein the culturing is performed for less than about 18 hours, and measuring expression levels of one or more histone modulating enzymes for changes in expression as compared to expression levels of one or more histone modulating enzymes from healthy controls.

In several embodiments, there is provided a multi-omic method of screening candidate therapeutics for treatment of a gastrointestinal disease, comprising separating an intestinal biopsy sample from a patient with at least one active gastrointestinal disease into a plurality of biopsy replicates, culturing the biopsy replicates in an artificial medium with each biopsy replicate in a tissue culturing environment, wherein the culturing is performed for less than about 18 hours, exposing a plurality of biopsy replicates to one of a plurality of candidate therapeutics to generate treatment biopsy replicates and maintaining at least one biopsy replicate in the artificial medium as an untreated biopsy replicate control, measuring, after the exposing, in one or more treatment biopsy replicates and an untreated biopsy control, two or more of the levels of one or more histone modification in the biopsy replicates, levels of one or more inflammatory cytokines/chemokines from the media in which each biopsy replicate is cultured, levels of one or more histone-modifying enzymes in the biopsy replicates, levels of one or more microRNAs in the biopsy replicates, and/or levels of one or more epitranscriptomic regulator in the biopsy replicates, comparing the levels measured, and determining, based on the comparing, that a candidate therapeutic is effective to treat a gastrointestinal disease if the candidate therapeutic induced changes in the levels measured in the respective treatment biopsy replicate are significantly different from the untreated biopsy replicate control.

There is also provided for herein a kit for screening candidate therapeutics for use in treating a gastrointestinal disease, comprising reagents for detecting, from an ex vivo culture of an intestinal biopsy, one or more of levels of one or more histone modification in the biopsy, levels of one or more inflammatory cytokines/chemokines from a media in which the biopsy is cultured, levels of one or more histone-modifying enzymes in the biopsy, levels of one or more microRNAs in the biopsy, and/or levels of one or more epitranscriptomic regulator in the biopsy; and instructions for use.

Some embodiments described herein relate to a method of screening candidate therapeutics for inflammatory bowel diseases, including ulcerative colitis and Crohn's Disease, using an ex vivo biopsy high throughput platform. Some embodiments include creating an ex vivo biopsy high throughput platform by i) receiving a biopsy sample from a patient with at least one active inflammatory bowel diseases, ii) separating the sample into a plurality of replicates, and iii) culturing the replicates in an artificial medium with each replicate in a tissue culturing environment. Some embodiments included adding a screening candidate therapeutics to the replicates in the ex vivo biopsy high throughput platform.

Some embodiments include measuring levels of one or more biomarkers within 12 hours in a biopsy treated with a screening candidate therapeutics in comparison to the levels of corresponding biomarkers in a replicate of the same biopsy that is untreated (no screening candidate therapeutics treatment). The biomarkers measured in the ex vivo biopsy high throughput platform may include, but are not limited to, the mRNA and protein levels of selected cytokines and chemokines, including TNF-alpha (TNFA), interleukin-1b (IL1B), IL1RA, IL2, IL4, IL5, IL6, IL7, IL8, IL10, IL12, IL13, IL15, IL17A, IL17E, IL33, GM-CSF, G-CSF, ICAM1, MCP-1, IFNgamma. MIP1A, MIP1B, CCL5/RANTES, CCL11, CD40, CX3CL1, CXCL1, CXCL2, CXCL10, TREM1, MIP3A and MIP3B. The biomarkers measured in the ex vivo biopsy high throughput platform could be the protein levels of the following histone modifications: H3K9me1/2/3, H3K27me1/2/3, H3K4me1/2/3, H3K36me1/2/3, H3K79me1/2/3, H3K9ac, H3K14ac, H3K18acm H3K56ac, H3ser10P, H3ser28P, total H3, H4K5ac, H4K8ac, H4K12ac, H4K16ac, H4R3m2a, H4R3m2s, H4K20me1/2/3, H4ser1, total H4, H3S10ph and H3S28ph, and/or the related histone-modifying genes (ASHIL, DOT1L, EHMT1, G9A/EHMT2,EZH1, EZH2, BMI1, SUZ12, MLL1-5, NSD1, PRDM2, SETD1-9, SETDB1/2, SETMAR, SMYD1-5, SUV39H1/2, SUV420H1/2, PRMT1-7, KDM1A/B, KMD2A/B, KMD3A/B, JMJDIC, KMD4A/B/C/D, KDM5A/B/C/D, KDM6A/B, GCN5, PCAF, HBO1,p300, CBP, SRC-1, ACTR, TIF2, TAF1, TFIIIC, HDAC1-11, SIRT1-7). The biomarkers measured in the ex vivo biopsy high throughput platform could be the expression levels of one or more mature microRNAs and/or the levels of epitranscriptomic regulators, including methyltransferases (METTL3, METTL14, METTL1, METTL6, METTL13, METTL16, NSUN2, NSUN3, NSUN4, TRMT2A, TRMT6, TRMT11, TRMT12, TRMT16B, BCDIN3D, KIAA1456, HENMTI, FBL, DIMT1), demethylases (ALKBH3, ALKBH5, FTO), readers (YTHDC2, YTHDF2) and deaminases (ADAR1, ADAR2).

Technologies used to evaluate the biomarkers measured in the ex vivo biopsy high throughput platform include, but are not limited to, multiplex ELISA, western blot, HPLC, LC-MS/MS, MALDI-TOF-MS, real-time PCR, RNA-sequencing, small RNA-sequencing, gene expression microarrays, nanostring profiling, ATAC-sequencing, ChIP-sequencing, chromatin-associated RNA-sequencing, and/or DNA methyl-sequencing.

In some embodiments, the biopsy sample is obtained by colonoscopy or sigmoidoscopy.

In some embodiments, the biopsy sample is from the microscopically diseased area of the intestinal mucosa of a patient. In some embodiments, the biopsy sample is from the gastrointestinal tract, for example, the small or large intestine, duodenum, stomach, esophagus of a patient. In some embodiments, the biopsy sample is from a patient with active Ulcerative colitis or Crohn's Disease, celiac disease, Irritable Bowel Syndrome (IBS), or esophagitis, or combinations of such maladies. In some embodiments, the biopsy sample is from a patient who is newly diagnosed or treated with steroids, immunomodulators and/or biologics.

In some embodiments, the biopsy sample can be separated into from 2 to 100 replicates.

In some embodiments, the artificial medium comprises L-alanyl-L-glutamine dipeptide, which is a dipeptide substitute for L-glutamine. In several embodiments, the L-alanyl-L-glutamine dipeptide can be GlutaMAX™. In some embodiments, the concentration of L-alanyl-L-glutamine dipeptide is between 1 mM to 10 mM. In some embodiments, the concentration of L-alanyl-L-glutamine dipeptide is between about 2 and about 6 mM, including about 2.5 mM, about 3 mM, about 3.5 mM, about 4 mM, about 4.5 mM, about 5 mM, about 5.5 mM, and any concentration between those listed.

In some embodiments, the tissue culturing environment is a well in a tissue culture plate. In some embodiments, the tissue plate has 1-1000 wells. In some embodiments, the tissue plate is a 96-or 384-well plate.

Some embodiments relate to a therapeutic composition for treating inflammatory bowel diseases, comprising a chemical compound targeting a histone modifying factor. In some embodiments, the histone modifying factor can be ASH1L, DOT1L, EHMT1, G9A/EHMT2, EZH1, EZH2, BMI1, SUZ12, MLL1-5, NSD1, PRDM2, SETD1-9, SETDB 1/2, SETMAR, SMYD1-5, SUV39H1/2, SUV420H1/2, PRMT1-7, KDM1A/B, KMD2A/B, KMD3A/B, JMJDIC, KMD4A/B/C/D, KDM5A/B/C/D, KDM6A/B (JMJD3), GCN5, PCAF, HBO1, p300, CBP, SRC-1, ACTR, TIF2, TAF1, TFIIIC, HDAC1-11, and/or SIRT1-7.

Some embodiments relate to a therapeutic composition for treating inflammatory bowel diseases, comprising a chemical compound targeting a cytokine or chemokine, including TNF-alpha (TNFA), interleukin-1b (IL1B), ILIRA, IL2, IL4, IL5, IL6, IL7, IL8, IL10, IL12, IL13, IL15, IL17A, IL17E, IL33, GM-CSF, G-CSF, ICAM1, MCP-1, IFNgamma. MIP1A, MIP1B, CCL5/RANTES, CCL11, CD40, CX3CL1, CXCL1, CXCL2, CXCL10, TREM1, MIP3A, and/or MIP3B.

Some embodiments relate to a therapeutic composition for treating inflammatory bowel diseases, comprising a chemical compound targeting an epitranscriptomic regulator, including methyltransferases (METTL3, METTL14, METTL1, METTL6, METTL13, METTL16, NSUN2, NSUN3, NSUN4, TRMT2A, TRMT6, TRMT11, TRMT12, TRMT16B, BCDIN3D, KIAA1456, HENMT1, FBL, DIMT1), demethylases (ALKBH3, ALKBH5, FTO) readers (YTHDC2, YTHDF2) and/or deaminases (ADAR1, ADAR2).

Some embodiments relate to a therapeutic composition for treating inflammatory bowel diseases, comprising a miR-24-3p inhibitor.

In some embodiments, the miR-24-3p inhibitor is an antisense-miR-24-3p compound, which contains a sequence complementarity to miR-24-3p. The antisense-miR-24-3p compound can bind to miR-24-3p specifically and inhibit the function of miR-24-3p. In some embodiments, the antisense-miR-24-3p compound has the sequence of SEQ ID NO: 1 (5-CTG^{m}CTGAA^{m}CTGAG^{m}CCA-3) or SEQ ID NO: 2 (5-CTGCTGAACTGAGCCA-3). In several embodiments, antisense-miR-24-3p compound has the sequence of SEQ ID NO: 3 (5-CTG^{m}CTGAA^{m}CTGAG^{m}CCA-3; bases 1-16 comprise phosphorothioate linkages, bases 1-3, 10-11, and 15-16 comprise locked nucleic acids, and the cytosine at position 4, 9, and 14comprises a methyl group (e.g., 5-methylcytosine)) or SEQ ID NO: 4 (5-CTGCTGAACTGAGCCA-3; bases 1-16 comprise phosphorothioate linkages and bases 1-2, and 14-16 comprise locked nucleic acids).

Some embodiments relate to a high-throughput method of screening an ex vivo biopsy for chromatin modifications associated with an inflammatory bowel disease. The high-throughput method include one or more of the following steps: a) receiving a biopsy sample from a patient with at least one active inflammatory bowel disease, b) separating the sample into a plurality of replicates, c) culturing the replicates in an artificial medium with each replicate in a tissue culturing environment, wherein the culturing is performed for less than about 18 hours, and d) measuring expression levels of one or more histone modulating enzymes for changes in expression as compared to expression levels of one or more histone modulating enzymes from healthy controls. In some embodiments, the replicates are cultured for about 4, about 6, or about 12 hours.

Some embodiments relate to a multi-omic method of screening candidate therapeutics for treatment of an inflammatory bowel disease.

In some embodiments, a multi-omic method can include one or more of the following steps:
a. receiving a biopsy sample from a patient with at least one active inflammatory bowel disease;
b. separating the sample into a plurality of biopsy replicates;
c. culturing the biopsy replicates in an artificial medium with each biopsy replicate in a tissue culturing environment, wherein the culturing is performed for less than about 18 hours;
d. exposing a plurality of biopsy replicates to one of a plurality of candidate therapeutics to generate treatment biopsy replicates and maintaining at least one biopsy replicate in the artificial medium as an untreated biopsy replicate control;
e. measuring the following at a first time-point during the culturing and after the exposure of step d:
   i. levels of one or more histone modification in each of the biopsy replicates,
   ii. levels of one or more inflammatory cytokines/chemokines from the media in which each biopsy replicate is cultured,
   iii. levels of one or more histone-modifying enzymes in each of the biopsy replicates,
   iv. levels of one or more microRNAs in each of the biopsy replicates, and/or
   v. levels of one or more epitranscriptomic regulator in each of the biopsy replicates;
f. comparing the values of
   i. the one or more histone modification levels from each treatment biopsy replicate to the one or more histone modification levels from the untreated biopsy replicate control,
   ii. the one or more inflammatory cytokine from each treatment biopsy replicate to one or more inflammatory cytokine levels from the untreated biopsy replicate control,
   iii. the one or more histone-modifying enzyme levels from each treatment biopsy replicate to one or more histone-modifying enzyme levels from the untreated biopsy replicate control,
   iv. the one or more epitranscriptomic regulator levels from each treatment biopsy replicate to one or more epitranscriptomic regulator levels from the untreated biopsy replicate control, and/or
   v. the one or more microRNA levels from each treatment biopsy replicate to one or more microRNA levels from the untreated biopsy replicate control; and
g. determining, based on the comparison of step f that a candidate therapeutic is effective to treat an inflammatory bowel disease if the candidate therapeutic induced changes in the one or more histone modification, histone-modifying enzyme, microRNA, epitrascriptomic regulator levels, and the one or more inflammatory cytokine levels such that the one or more histone modification, histone-modifying enzyme, microRNA, epitrascriptomic regulator levels and the one or more inflammatory cytokine levels measure in the respective treatment biopsy replicate are significantly different from the untreated biopsy replicate control.

In some embodiments, the one or more histone modification levels include measurements related to the methylation or demethylation of a histone. In some embodiments, the one or more histone modification levels include measurements of H3K9me1/2/3, H3K27me1/2/3, H3K4me1/2/3, H3K36me1/2/3, H3K79me1/2/3, H3K9ac, H3K14ac, H3K18acm H3K56ac, H3ser10P, H3ser28P, total H3, H4K5ac, H4K8ac, H4K12ac, H4K16ac, H4R3m2a, H4R3m2s, H4K20me1/2/3, H4ser1, total H4, H3S10ph and/or H3S28ph. In some embodiments, the one or more inflammatory cytokine measured include TNF-alpha (TNFA), interleukin-1b (IL1B), IL1RA, IL2, IL4, IL5, IL6, IL7, IL8, IL10, IL12, IL13, IL15, IL17A, IL17E, IL33, GM-CSF, G-CSF, ICAMI, MCP-1, IFNgamma. MIP1A, MIP1B, CCL5/RANTES, CCL11, CD40, CX3CL1, CXCL1, CXCL2, CXCL10, TREM1, MIP3A, and/or MIP3B.

In some embodiments, the candidate therapeutics are inhibitors of one or more of epigenetic factors ASH1L, DOT1L, EHMT1, G9A/EHMT2,EZH1, EZH2, BMI1, SUZ12, MLL1-5, NSD1, PRDM2, SETD1-9, SETDB1/2, SETMAR, SMYD1-5, SUV39H1/2, SUV420H1/2, PRMT1-7, KDM1A/B, KMD2A/B, KMD3A/B, JMJDIC, KMD4A/B/C/D, KDM5A/B/C/D, KDM6A/B (JMJD3), GCN5, PCAF, HBO1,p300, CBP, SRC-1, ACTR, TIF2, TAF1, TFIIIC, HDAC1-11, SIRT1-7, METTL3, METTL14, METTL1, METTL6, METTL13, METTL16, NSUN2, NSUN3, NSUN4, TRMT2A, TRMT6, TRMT11, TRMT12, TRMT16B, BCDIN3D, KIAA1456, HENMT1, FBL, DIMT1, ALKBH3, ALKBH5, FTO, YTHDC2, and YTHDF2.

In some embodiments, the candidate therapeutic is an antisense-miR-24-3p compound. In some embodiments, the antisense-miR-24-3p compound has the sequence of SEQ ID NO: 1 (5-CTG^{m}CTGAA^{m}CTGAG^{m}CCA-3) or SEQ ID NO: 2 (5-CTGCTGAACTGAGCCA-3). In several embodiments, the antisense compounds comprise one or more modifications, such as locked nucleic acids (LNAs), methyl substitution at one or more of the ribonucleotides, and/or one or a plurality of phosphorothioate linkages along the RNA.

These and other features, aspects, and advantages of the present invention will become better understood with reference to the following description and appended claims.

### BRIEF DESCRIPTION OF DRAWINGS

FIG. 1 shows data related to cell viability of IBD colonic explants during transfer using different concentrations of GlutaMAX™ (L-alanyl-L-glutamine dipeptide) in transfer media.

FIG. 2 shows data related to cell viability of colonic explant cultures in different time points (4-24 hours).

FIG. 3 shows data related to G9A and JMJD3 mRNA expression in colonic biopsies from healthy control (n=10) and UC patients with active disease (n=12).

FIG. 4 shows data related to IL-1beta (IL1B) and TNF-alpha (TNFA) expression levels in supernatant of colonic explant culture treated with 10 uM GSK-J4, JIB-04, BIX01294, UNC0642 and UNC1999 for 12 h.

FIGS. 5A-5B show data related to quantification of various epigenetic modifications. FIG. 5A shows quantification of H3K9me2 modifications and FIG. 5B shows quantification of H3K27me3 modifications in the ex vivo biopsy high throughput platform treated with BIX01294, UNC0642, GSK-J4, JIB-04 and UNC1999 epigenetic drugs.

FIGS. 6A-6B show data related to quantification of various cytokines and micro RNAs. FIG. 6A shows IL1B shows TNFA levels in supernatant of a UC colonic explant culture treated with 1 uM miR-24-A and miR-24-B compounds for 12 h. FIG. 6B shows MiR-24-3p expression levels in a UC colonic explant culture treated with 1 uM miR-24-A and miR-24-B compounds for 12 h.

### DETAILED DESCRIPTION

In the Summary Section above and the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. As used herein, “comprises” means “includes.” Thus, “comprising A or B,” means “including A, B, or A and B,” without excluding additional elements. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. Unless otherwise specified, the definitions provided herein control when the present definitions may be different from other possible definitions.

A “therapeutic” refers to a treatment, therapy, or drug.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance.

“Ex vivo” means that which takes place outside an organism. In science, ex vivo refers to experimentation or measurements done in or on tissue from an organism in an external environment with minimal alteration of natural conditions.

The term “omes” or “ome” refers to a genome, transcriptome, epitranscriptome, epigenome, immunome, microbiome, proteome and metabolome. Similarly, the term “omics” refers to genomics, transcriptomics, epitranscriptomics, epigenomics, immunomics, microbiomics, proteomics, or metabolomics.

A “genome” is the genetic material of an organism. It consists of DNA. The genome includes both the genes and the noncoding DNA, as well as mitochondrial DNA and chloroplast DNA. The study of the genome is called genomics.

A “transcriptome” is the set of all RNA molecules, including coding and non-coding RNAs. RNA is the main carrier of genetic information that is responsible for the process of converting DNA into an organism's phenotype. In addition to the protein-coding mRNA transcripts, the transcriptome included the long non-coding RNAs (>200 nucleotides long), the transfer RNAs (tRNAs), the microRNAs, the small interfering RNAs (siRNAs), the small nucleolar RNAs (snoRNAs), the piwi-interacting RNAs (piRNAs) and the enhancer RNAs (eRNAs). The study of the transcriptome is called transcriptomics.

An “epitranscriptome” includes all the chemical modifications that affect RNA function. Specifically, m⁶A describes the methylation of the nitrogen at position 6 in the adenosine base within mRNA, N1-methyladenosine is a modified nucleoside in which a methyl group is added to N1 of the adenosine base, 5-methylcytosine, commonly abbreviated as “m⁵C”, is a chemical modification identified in tRNAs, Adenosine-to-Inosine (A-to-I) modifications, queuine (Q) is a modified nucleotide at position 34 in tRNA, 2′-O-methylation refers to the methylation of the 2′ hydroxyl group of the ribose within an RNA nucleotide, and pseudouridine (Ψ, 5-ribosyluracil) is another RNA modification. The study of the epitranscriptome is called epitranscriptomics.

An “epigenome” consists of a record of the chemical changes to the DNA and histone proteins of an organism; these changes can be passed down to an organism's offspring via transgenerational stranded epigenetic inheritance. Changes to the epigenome can result in changes to the structure of chromatin and changes to the function of the genome. The epigenome is involved in regulating gene expression, development, tissue differentiation, and suppression of transposable elements. Unlike the underlying genome, which remains largely static within an individual, the epigenome can be dynamically altered by environmental conditions. The study of the epigenome is called epigenomics. “Epigenetic factors” are factors other than an individual's DNA sequence that involve in genetic control.

An “immunome” is the set of genes and proteins that constitute the immune system, excluding those that are widespread in other cell types, and not involved in the immune response itself. It is further defined as the set of peptides derived from the proteome that interact with the immune system. The study of the immunome is called immunomics.

A “microbiome” is the aggregate of all microbiota that reside on or within an organism's tissues and biofluids along with the corresponding anatomical sites in which they reside, including the skin, mammary glands, placenta, seminal fluid, uterus, ovarian follicles, lung, saliva, oral mucosa, conjunctiva, biliary tract, and gastrointestinal tract. The study of the microbiome is called microbiomics.

A “proteome” is the aggregate of all proteins expressed in a cell, tissue or organism. The human proteome consists of ˜92,000 proteins, while the plasma proteome includes 10,500 plasma proteins. Mass spectrometry is one of the key methods to study and characterize the proteome. The study of the proteome is called proteomics.

A “metabolome” is the aggregate of small-molecule chemicals that are naturally produced by an organism, such as amino acids, organic acids, nucleic acids, fatty acids, amines, sugars and vitamins. The metabolome reflects the interaction between an organism's genome and its environment. The study of the metabolome is called metabolomics.

The term “signature” refers to a group of genes, proteins, cytokines, or other factors in a cell or tissue whose combined expression pattern is uniquely characteristic of the cell of tissue. For example, disease signature is usually a list of genes, within a specific tissue, that are up or down regulated when compared to healthy controls.

The term “cytokine” refers to a broad and loose category of small proteins (˜5-20 kDa) involved in cell signaling. Cytokines are peptides and cannot cross the lipid bilayer of cells to enter the cytoplasm. Cytokines have been shown to be involved in autocrine, paracrine and endocrine signaling as immunomodulating agents. Cytokines include chemokines, interferons, interleukins, lymphokines, and tumor necrosis factors. Cytokines are produced by a broad range of cells, including immune cells like macrophages, B lymphocytes, T lymphocytes and mast cells, as well as endothelial cells, fibroblasts, and various stromal cells; a given cytokine may be produced by more than one type of cell. They act through cell surface receptors and are especially important in the immune system; cytokines modulate the balance between humoral and cell-based immune responses, and they regulate the maturation, growth, and responsiveness of particular cell populations.

“Chemokines” are a family of small cytokines, or signaling proteins secreted by cells. Their name is derived from their ability to induce directed chemotaxis, which is the movement of an organism in response to a chemical stimulus, in nearby responsive cells; they are chemotactic cytokines.

“Small RNA” are <200 nt (nucleotide) in length, and are usually non-coding RNA molecules. RNA silencing is often a function of these molecules, with the most common and well-studied example being RNA interference (RNAi), in which endogenously expressed microRNA (miRNA) or exogenously derived small interfering RNA (siRNA) induces the degradation of complementary messenger RNA.

A “microRNA” (abbreviated miRNA) is a small non-coding RNA molecule (containing about 22 nucleotides) found in plants, animals and some viruses, that functions in RNA silencing and post-transcriptional regulation of gene expression. miRNAs function via base-pairing with complementary sequences within mRNA molecules. As a result, these mRNA molecules are silenced, by one or more of the following processes: (1) Cleavage of the mRNA strand into two pieces, (2) Destabilization of the mRNA through shortening of its poly (A) tail, and (3) Less efficient translation of the mRNA into proteins by ribosomes. miRNAs resemble the small interfering RNAs (siRNAs) of the RNA interference (RNAi) pathway, except miRNAs derive from regions of RNA transcripts that fold back on themselves to form short hairpins, whereas siRNAs derive from longer regions of double-stranded RNA.

“Antisense molecules” include several classes of oligonucleotide molecules that contain sequence complementarity to target RNA molecules, such as mRNA, viral RNA, or other RNA species, and that inhibit the function of their target RNA after sequence-specific binding.

### Various Embodiments

Some embodiments described herein relate to a method of screening candidate therapeutics for inflammatory bowel diseases. Inflammatory bowel diseases include, but are not limited to, ulcerative colitis and Crohn's Disease. Provided for herein is an ex vivo biopsy high throughput platform that facilitates screening of candidate therapeutics. Some embodiments include creating an ex vivo biopsy high throughput platform by i) receiving a biopsy sample from a patient with at least one active inflammatory bowel diseases, ii) separating the sample into a plurality of replicates, and iii) culturing the replicates in an artificial medium (thus each replicate is incubated in a tissue culturing environment). Some embodiments included adding at least one candidate therapeutic to the replicates in the ex vivo biopsy high throughput platform as well as maintaining at least one control replicate.

Some embodiments include measuring levels of one or more biomarkers within 12 hours in a colonic biopsy treated with a screening candidate therapeutics in comparison to the levels of corresponding biomarkers in a replicate of the same colonic biopsy that is untreated (e.g., not contacted with a screening candidate therapeutic). The biomarkers measured in the ex vivo biopsy high throughput platform could, depending on the embodiment, be mRNA and/or protein levels of one or more selected cytokines and chemokines, including, but not limited to, TNF-alpha (TNFA), interleukin-1b (IL1B), IL1RA, IL2, IL4, IL5, IL6, IL7, IL8, IL10, IL12, IL13, IL15, IL17A, IL17E, IL33, GM-CSF, G-CSF, ICAM1, MCP-1, IFNgamma. MIP1A, MIP1B, CCL5/RANTES, CCL11, CD40, CX3CL1, CXCL1, CXCL2, CXCL10, TREM1, MIP3A and/or MIP3B. The biomarkers measured in the ex vivo biopsy high throughput platform could be, depending on the embodiment, the protein levels of one or more of the following histone modifications: H3K9me1/2/3, H3K27me1/2/3, H3K4me1/2/3, H3K36me1/2/3, H3K79me1/2/3, H3K9ac, H3K14ac, H3K18acm H3K56ac, H3ser10P, H3ser28P, total H3, H4K5ac, H4K8ac, H4K12ac, H4K16ac, H4R3m2a, H4R3m2s, H4K20me1/2/3, H4ser1, total H4, H3S10ph and H3S28ph, and/or the related histone-modifying genes (ASH1L, DOT1L, EHMT1, G9A/EHMT2,EZH1, EZH2, BMI1, SUZ12, MLL1-5, NSD1, PRDM2, SETD1-9, SETDB1/2, SETMAR, SMYD1-5, SUV39H1/2, SUV420H1/2, PRMT1-7, KDM1A/B, KMD2A/B, KMD3A/B, JMJDIC, KMD4A/B/C/D, KDM5A/B/C/D, KDM6A/B, GCN5, PCAF, HBO1,p300, CBP, SRC-1, ACTR, TIF2, TAF1, TFIIIC, HDAC1-11, SIRT1-7). The biomarkers measured in the ex vivo biopsy high throughput platform could, depending on the embodiment, be the expression levels of one or more mature microRNAs and/or the levels of epitranscriptomic regulators, including, but not limited to, methyltransferases (e.g., METTL3, METTL14, METTL1, METTL6, METTL13, METTL16, NSUN2, NSUN3, NSUN4, TRMT2A, TRMT6, TRMT11, TRMT12, TRMT16B, BCDIN3D, KIAA1456, HENMT1, FBL, DIMT1), demethylases (e.g., ALKBH3, ALKBH5, FTO), readers (e.g., YTHDC2, YTHDF2) and deaminases (e.g., ADAR1, ADAR2).

Technologies used to evaluate the biomarkers measured in the ex vivo biopsy high throughput platform include, but are not limited to, multiplex ELISA, western blot, HPLC, LC-MS/MS, MALDI-TOF-MS, real-time PCR, RNA-sequencing, small RNA-sequencing, gene expression microarrays, nanostring profiling, ATAC-sequencing, ChIP-sequencing, chromatin-associated RNA-sequencing, and/or DNA methyl-sequencing.

In some embodiments, the colon biopsy sample is obtained by colonoscopy or sigmoidoscopy. A colon biopsy is the removal and examination of a tissue sample from the colon. It is a diagnostic procedure used to determine whether any of the tissue cells are cancerous or precancerous, or inflammatory. Colonoscopy and sigmoidoscopy are very similar but different procedures. A colonoscopy is an exam used to detect changes or abnormalities in the large intestine (colon) and rectum. During a colonoscopy, a long, flexible tube (colonoscope) is inserted into the rectum. A video camera at the tip of the tube allows the doctor to view the inside of the entire colon and evaluate the health of the entire large intestine. On the other hand, a sigmoidoscopy is less invasive because only the lower part of the colon is looked at. Typically, pieces of tissue will be taken as samples to check for any abnormal cell changes.

In some embodiments, the biopsy sample is from the microscopically diseased, or otherwise unhealthy, area of the intestinal mucosa of a patient. The intestinal mucosa is the inner lining of the intestinal tract.

In some embodiments, the biopsy sample is from the small or large intestine of a patient. Depending on the severity, inflammatory bowel diseases may cause inflammation at different locations of the lining of one's digestive tract, such as small intestine, large intestine, or rectum. Depending on the location of inflammation, the biopsy sample may be taken from the small or large intestine.

In some embodiments, the biopsy sample is from a patient with active or suspected Ulcerative colitis or Crohn's Disease. In some embodiments, the biopsy sample is from a patient who is newly diagnosed or treated with steroids, immunomodulators and/or biologics.

In some embodiments, the biopsy sample can be separated into from 2 to 100 replicates. The number of replicates depends on the size of the biopsy sample and the variables to be tested. The biopsy sample can be separated into from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 replicates. In other embodiments, larger numbers of replicates are generated, either by dividing the biopsy into smaller pieces, or for example, obtaining an additional biopsy sample from the patient.

In some embodiments, the artificial medium comprises L-alanyl-L-glutamine dipeptide, which is a dipeptide substitute for L-glutamine. In several embodiments, the L-alanyl-L-glutamine dipeptide is GlutaMAX™. In some embodiments, the concentration of L-alanyl-L-glutamine dipeptide is between 1 mM to 10 mM. In some embodiments, the concentration of L-alanyl-L-glutamine dipeptide is between about 2 and about 6 mM, including about 2.5 mM, about 3 mM, about 3.5 mM, about 4 mM, about 4.5 mM, about 5 mM, about 5.5 mM, and any concentration between those listed. In several embodiments, the concentration of L-alanyl-L-glutamine dipeptide is 4 mM.

In some embodiments, the tissue culturing environment is a well in a tissue culture plate. In some embodiments, the tissue plate has 1-1000 wells. In some embodiments, the tissue plate is a 96- or 384-well plate. In several embodiments, other vessels are used, for example 0.5-1.5 ml conical tubes, as a non-limiting example.

Some embodiments relate to a therapeutic composition for treating inflammatory bowel diseases, comprising a chemical compound targeting a histone modifying factor. In some embodiments, the histone modifying factor is one or more of ASH1L, DOT1L, EHMT1, G9A/EHMT2, EZH1, EZH2, BMI1, SUZ12, MLL1-5, NSD1, PRDM2, SETD1-9, SETDB1/2, SETMAR, SMYD1-5, SUV39H1/2, SUV420H1/2, PRMT1-7, KDM1A/B, KMD2A/B, KMD3A/B, JMJDIC, KMD4A/B/C/D, KDM5A/B/C/D, KDM6A/B (JMJD3), GCN5, PCAF, HBO1, p300, CBP, SRC-1, ACTR, TIF2, TAF1, TFIIIC, HDAC1-11, or SIRT1-7.

Some embodiments relate to a therapeutic composition for treating inflammatory bowel diseases, comprising a chemical compound targeting a cytokine or chemokine, including one or more of TNF-alpha (TNFA), interleukin-1b (IL1B), IL1RA, IL2, IL4, IL5, IL6, IL7, IL8, IL10, IL12, IL13, IL15, IL17A, IL17E, IL33, GM-CSF, G-CSF, ICAM1, MCP-1, IFNgamma. MIPIA, MIP1B, CCL5/RANTES, CCL11, CD40, CX3CL1, CXCL1, CXCL2, CXCL10, TREM1, MIP3A, and MIP3B.

Some embodiments relate to a therapeutic composition for treating inflammatory bowel diseases, comprising a chemical compound targeting an epitranscriptomic regulator, including, but not limited to methyltransferases (e.g., METTL3, METTL14, METTL1, METTL6, METTL13, METTL16, NSUN2, NSUN3, NSUN4, TRMT2A, TRMT6, TRMT11, TRMT12, TRMT16B, BCDIN3D, KIAA1456, HENMT1, FBL, DIMT1), demethylases (e.g., ALKBH3, ALKBH5, FTO) readers (e.g., YTHDC2, YTHDF2) and deaminases (e.g., ADAR1, ADAR2).

Recently, epigenetic and transcriptomic factors, such as small RNA molecules (called microRNAs), have been implicated in IBD pathogenesis. According to some embodiments, antisense molecules that block the function of microRNAs are provided and serve to reduce, treat, cure, or otherwise ameliorate IBD and other chronic inflammatory diseases.

Accordingly, some embodiments relate to a therapeutic composition for treating inflammatory bowel diseases, comprising a miR-24-3p inhibitor. miR-24-3p is a microRNA belonging to the miR-24 microRNA precursor family, which is a small non-coding RNA molecule that regulates gene expression. miR-24 is transcribed as ˜70 nucleotide precursor and subsequently processed by the Dicer enzyme to give a mature ˜22 nucleotide product. miR-24-3p is extensively expressed in various tissues and plays an important role in various physiological or pathological conditions. In some embodiments, the miR-24-3p inhibitor is an antisense-miR-24-3p compound, which contains at least some degree of sequence complementarity to miR-24-3p. For example, in several embodiments, the antisense miR24-3p compound has at least about 50%, at least about 60%, at least about 70%, at least about 75%, at least about 80%, at least about 85%, at least about 90%, or at least about 95% sequence complementarity to miR-24-3p (or such complementarity within an internal region of the mature or immature miR-24). In several embodiments, the antisense-miR-24-3p compound can bind to miR-24-3p specifically and inhibit, at least partially, the function of miR-24-3p. In some embodiments, the antisense-miR-24-3p compound has the sequence of SEQ ID NO: 1 (5-CTGMCTGAAMCTGAGMCCA-3) or SEQ ID NO: 2 (5-CTGCTGAACTGAGCCA-3).

Some embodiments relate to a high-throughput method of screening an ex vivo biopsy for chromatin modifications associated with an inflammatory bowel disease. The high-throughput method, according to several embodiments, includes one or more of the following steps: a) receiving a biopsy sample from a patient with at least one active, or suspected, inflammatory bowel disease, b) separating the sample into a plurality of replicates, c) culturing the replicates in an artificial medium with each replicate in an independent tissue culturing environment, and d) measuring one or more metrics as provided for herein. In several embodiments, expression levels of one or more histone modulating enzymes are quantified to identify changes in expression as compared to expression levels of one or more histone modulating enzymes from healthy controls. In several embodiments, the culturing is performed for less than about 18 hours. In some embodiments, the replicates are cultured for 4, 6, or 12 hours.

Some embodiments relate to a multi-omic method of screening candidate therapeutics for treatment of an inflammatory bowel disease. “Multi-omic” is a term referred to any factor related to genome, transcriptome, epitranscriptome, epigenome, immunome, proteome, metabolome or microbiome. The current therapeutics, such as TNF-alpha antibodies and other cytokine receptor inhibitors do not have high efficacy in treating IBD patients (Fiocchi C, Pharmacol. Res., 2020), partially due to the absence of a biomarker assay able to predict response to these drugs. Although most efforts have been focused on identifying a cytokine/chemokine-related biomarker for IBD drugs, it is now understood that IBDs are multifactorial diseases developed due to deregulation of multiple “omes”, such as the genome, epigenome, immunome and microbiome (de Souza H S P et al., Nat. Rev. Gastroenterol. Hepatol., 2017). Thus, as provided for herein, it is important to develop a comprehensive methodology and platform able to capture multiple “ome” alterations in addition to cytokine production levels.

In some embodiments, a multi-omic method comprises, consists of, or consists essentially of one or more of the following steps:
a. receiving a biopsy sample from a patient with at least one active or suspected inflammatory bowel disease;
b. separating the sample into a plurality of biopsy replicates;
c. culturing the biopsy replicates in an artificial medium with each biopsy replicate in a tissue culturing environment;
d. exposing a plurality of biopsy replicates to an individual one of a plurality of candidate therapeutics to generate treatment biopsy replicates and maintaining at least one biopsy replicate in the artificial medium as an untreated biopsy replicate control;
e. measuring the following at a first time-point during the culturing and after the exposure of step d at least one of:
   vi. levels of one or more histone modification in each of the biopsy replicates,
   vii. levels of one or more inflammatory cytokines/chemokines from the media in which each biopsy replicate is cultured,
   viii. levels of one or more histone-modifying enzymes in each of the biopsy replicates,
   ix. levels of one or more microRNAs in each of the biopsy replicates, and/or
   x. levels of one or more epitranscriptomic regulator in each of the biopsy replicates;
f. comparing the values of
   vi. the one or more histone modification levels from each treatment biopsy replicate to the one or more histone modification levels from the untreated biopsy replicate control,
   vii. the one or more inflammatory cytokine from each treatment biopsy replicate to one or more inflammatory cytokine levels from the untreated biopsy replicate control,
   viii. the one or more histone-modifying enzyme levels from each treatment biopsy replicate to one or more histone-modifying enzyme levels from the untreated biopsy replicate control,
   ix. the one or more microRNA levels from each treatment biopsy replicate to one or more microRNA levels from the untreated biopsy replicate control; and
   x. the one or more epitranscriptomic regulator levels from each treatment biopsy replicate to one or more epitranscriptomic regulator levels from the untreated biopsy replicate control, and/or
g. determining, based on the comparison of step f that a candidate therapeutic is effective to treat an inflammatory bowel disease if the candidate therapeutic induced changes in the one or more histone modification, histone-modifying enzyme, microRNA, epitrascriptomic regulator levels, and the one or more inflammatory cytokine levels such that the one or more histone modification, histone-modifying enzyme, microRNA, epitrascriptomic regulator levels and the one or more inflammatory cytokine levels measure in the respective treatment biopsy replicate are significantly different from the untreated biopsy replicate control.

In some embodiments, the one or more histone modification levels include measurements related to the methylation or demethylation of a histone. In some embodiments, the one or more histone modification levels include measurements of H3K9me1/2/3, H3K27me1/2/3, H3K4me1/2/3, H3K36me1/2/3, H3K79me1/2/3, H3K9ac, H3K14ac, H3K18acm H3K56ac, H3ser10P, H3ser28P, total H3, H4K5ac, H4K8ac, H4K12ac, H4K16ac, H4R3m2a, H4R3m2s, H4K20me1/2/3, H4ser1, total H4, H3S10ph and/or H3S28ph. In some embodiments, the one or more inflammatory cytokine measured include TNF-alpha (TNFA), interleukin-1b (IL1B), IL1RA, IL2, IL4, IL5, IL6, IL7, IL8, IL10, IL12, IL13, IL15, IL17A, IL17E, IL33, GM-CSF, G-CSF, ICAMI, MCP-1, IFNgamma. MIP1A, MIP1B, CCL5/RANTES, CCL11, CD40, CX3CL1, CXCL1, CXCL2, CXCL10, TREM1, MIP3A, and/or MIP3B.

In some embodiments, the candidate therapeutics are inhibitors of one or more of epigenetic factors ASH1L, DOT1L, EHMT1, G9A/EHMT2,EZH1, EZH2, BMI1, SUZ12, MLL1-5, NSD1, PRDM2, SETD1-9, SETDB1/2, SETMAR, SMYD1-5, SUV39H1/2,SUV420H1/2, PRMT1-7, KDM1A/B, KMD2A/B, KMD3A/B, JMJDIC, KMD4A/B/C/D, KDM5A/B/C/D, KDM6A/B (JMJD3), GCN5, PCAF, HBO1,p300, CBP, SRC-1, ACTR, TIF2, TAF1, TFIIIC, HDAC1-11, SIRT1-7, METTL3, METTL14, METTL1, METTL6, METTL13, METTL16, NSUN2, NSUN3, NSUN4, TRMT2A, TRMT6, TRMT11, TRMT12, TRMT16B, BCDIN3D, KIAA1456, HENMT1, FBL, DIMT1, ALKBH3, ALKBH5, FTO, YTHDC2, and/or YTHDF2.

In some embodiments, the candidate therapeutic is an antisense-miR-24-3p compound. In some embodiments, the antisense-miR-24-3p compound has the sequence of SEQ ID NO: 1 (5-CTGMCTGAACTGAGMCCA-3) or SEQ ID NO: 2 (5-CTGCTGAACTGAGCCA-3).

### Example 1

This example describes non-limiting example of methods of creating an ex vivo biopsy high throughput platform.

Step 1: A biopsy was obtained from a macroscopically disease area of the inflammatory intestinal mucosa of an IBD patient. Samples can be from small or large intestine and the patient can be afflicted with, or suspected of having, UC or CD. The medium used to transport mucosal colonic biopsies from IBD patients was pre-cooled on ice, and comprised: Dulbecco's Modified Eagle Medium (DMEM) liquid (High Glucose), 4 mM L-alanyl-L-glutamine dipeptide, 1% penicillin/streptomycin and 30 mg/ml gentamicin. Tissue culture was initiated less than 1 hour after collection.

Step 2: Once in the laboratory, the biopsy was washed in Hank's Balanced Salt Solution buffer for a few minutes (e.g., 2-5 min) and cut with sterile scalpels to create biopsy pieces (approximately 2-5 um sections). Media comprising Dulbecco's Modified Eagle Medium (DMEM) liquid (High Glucose), 4 mM L-alanyl-L-glutamine dipeptide, 1% FBS, 1% non-essential amino acids, 1% Sodium Pyruvate, 1% penicillin/streptomycin, 30 mg/ml gentamicin and 5 ug/ml insulin-transferrin-selenium-X, was pre-warmed at 37° C. and added to the pieces of the biopsy. The average volume of media for each biopsy was 1.5 mL, which allows generation of 10 replicates in a 96 well plate (150 uL/well) or 60 replicates in a 384 well plate (25 uL/well). The plates then were cultured in a humidified chamber with 95% O₂ and 5% CO₂. The 96-and 384-well plates are two non-limiting embodiments of the platform assay disclosed herein, though additional embodiments of the high throughput platform disclosed herein can be used to predict drug responses by evaluating multi-omic signatures in other multi-well formats.

Step 3: Cell viability evaluation. To evaluate the effects of any drug using a colonic explant assay, the viability of the colonic explant in culture is examined. The experimental conditions related to the culture viability of the colonic explants were optimized by focusing on the media composition during the transfer of the biopsy from the patient biopsy collection site to the laboratory and the duration of culturing the explant.

CellTiter-Blue assay (Promega) was used to evaluate cell viability of the colonic explants. Briefly, the CellTiter-Blue reagent was added in the culture and the fluorescent signal was measured (560/590 nm) after 1 h of incubation in 37° C.

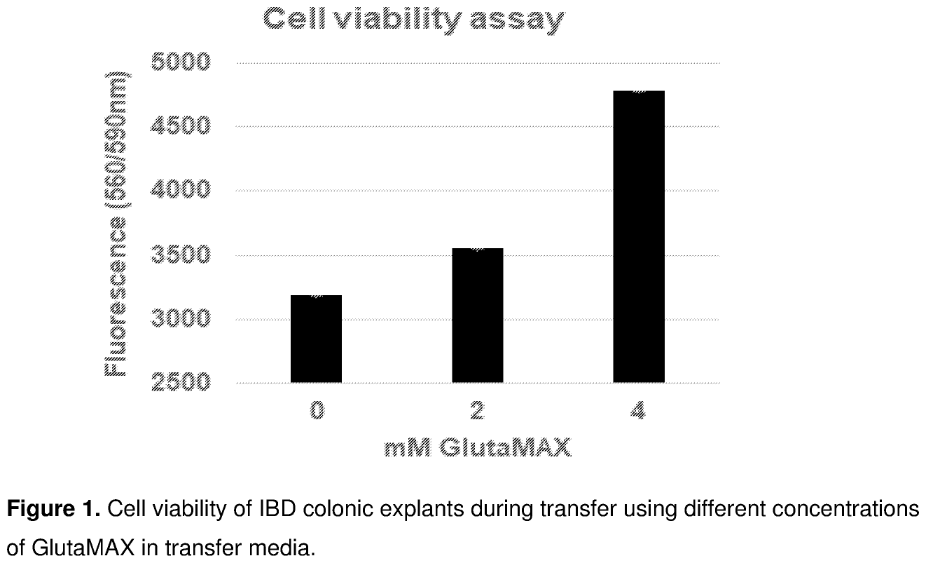

Colonic explants that were transferred into the lab in media (DMEM, 1% penicillin/streptomycin and 30 gentamicin) containing L-alanyl-L-glutamine dipeptide (GlutaMAX™ is one non-limiting example of such a dipeptide). Adding an L-alanyl-L-glutamine dipeptide into the transfer media resulted in substantial increase in cell viability (see FIG. 1). This is an unexpected enhancement of survival, given that such glutamine dipeptides are not traditionally included in transfer media. A concentration of 4 mM of L-alanyl-L-glutamine dipeptide was identified to have the highest cell viability (see FIG. 1). Thus, in several embodiments, a concentration of a L-alanyl-L-glutamine dipeptide between about 1 to about 10 mM is used, including about 1 to about 2 mM, about 2 to about 3 mM, about 3 to about 4 mM, about 4 to about 5 mM, about 5 to about 6 mM, about 6 to about 7 mM, about 7 to about 8 mM, about 8 to about 9 mM, about 9 to about 10 mM and any concentration therebetween, including endpoints. In several embodiments, other methods are used to prevent or reduce ammonia generation is used, which improves the health of the explants. In several embodiments, media is recirculated through a cationic exchanger, such as for example clinoptilolite. In several embodiments, a hydrophobic microporous module is used. In several embodiments, these are used optionally in connection with glutamate supplementation of the media (either in place of or addition to glutamine or a L-alanyl-L-glutamine dipeptide).

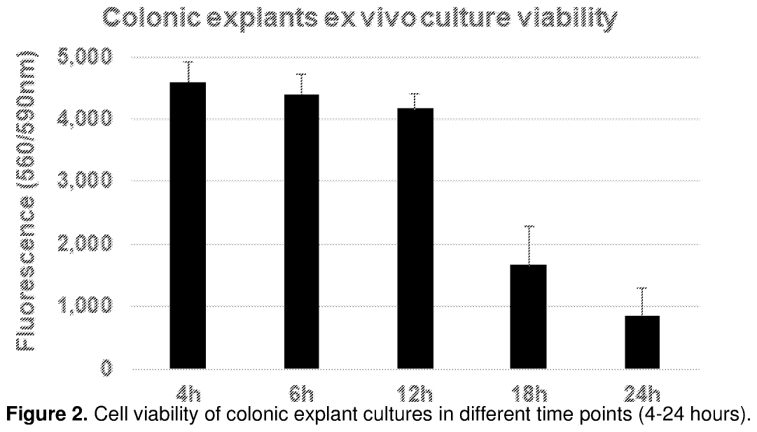

The “health status” of a colonic biopsy (e.g., whether the colonic explant and cells are healthy and not apoptotic or necrotic) was then evaluated and the appropriate time window was identified. The data in FIG. 2 showed that there was little cell death up to 12 hours culturing, confirming that the screening assay disclosed herein is reliable to perform any drug and/or molecular study, when the explant is in culture for up to about 12 hours (FIG. 2). As discussed herein, in several embodiments, other time frames, whether shorter or modestly longer, can be used.

Note when the explant remained 18 hours in culture there was a high degree of cell death. Thus, according to several embodiments, the effects of a new drug and the evaluation of a multi-omic signature in a colonic explant culture should ideally be examined less than 18 hours after the explant is plated and cultured (e.g., about 8-14 hours or about 12 hours, depending on the embodiment). Cytokine and other molecular changes detected at 18+ hours in culture could be significantly affected by the cell debris and cytokines secreted from dying colonocytes and may not be appropriate for a high throughput drug screen.

### Example 2

This example describes the use of non-limiting embodiments of the ex vivo biopsy high throughput platform for identifying Epigenome-Immunome multi-omic signature of response for small molecule chemical compounds.

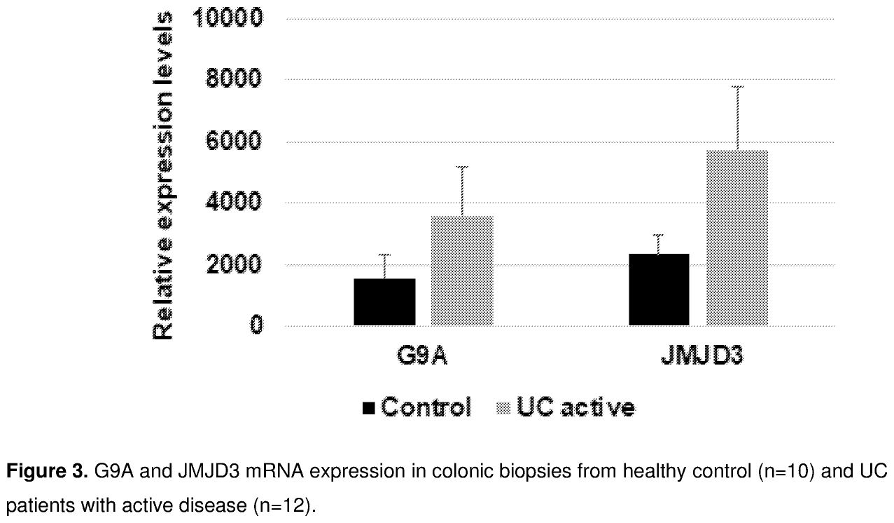

Previous studies have identified the importance of epigenetic alterations in IBD pathogenesis. Alterations at the level of DNA methylation and histone tails have been identified in IBD patients (Somineni H K et al., Gastroenterology, 2019; Sarmento O F et al. J Biol Chem, 2017). It is known that G9A is a histone methyltransferase that regulates histone 3 lysine 9 di-methylation (H3K9me2) levels (Tao H et al., Mol Cell Biochem, 2014), JMJD3 a histone demethylase regulating histone 3 lysine 27 tri-methylation (H3K27me3) levels (Agger K et al., Genes & Dev, 2009), and EZH2 a histone methyltransferase regulation H3K27me3 levels (Yamagishi M et al., Cell Reports, 2019). The role of EZH2 is already known in IBD pathogenesis and mice deficient in EZH2 develop colitis (Sarmento OF et al., J Biol Chem, 2017). However, the roles of G9A and JMJD3 are not clear. Here, as the data shown in FIG. 3, for the first time, that G9A and JMJD3 mRNA levels are increased in colonic biopsies from UC patients with active disease compared to healthy controls (FIG. 3). The data suggests that G9A and JMJD3 potentially have a pro-inflammatory role in IBD development, which makes them attractive therapeutic targets.

An embodiment of the ex vivo biopsy high throughput platform was used to assess the effects of G9A inhibitors BIX01294 (cat. No 3364, Tocris) and UNC0642 (cat. No 5132, Tocris), JMJD3 inhibitors GSK-J4 (cat. No 4594, Tocris) and JIB-04 (cat. No 4972, Tocris), and an EZH2 inhibitor UNC1999 (cat. No 4904, Tocris) (10 uM) on a multi-omic response, evaluating cytokine production levels and global chromatin H3K9me2 and H3K27me3 levels in UC active disease colonic explants.

Specifically, a UC active disease colonic biopsy was processed as described in Example 1 and separated into 6 replicates in a 96-well plate. One well was used as control and the rest were treated with each compound at the concentration of 10 uM. Twelve hours later, 50 uL of supernatant was collected to evaluate IL-1B (#KHC0011, ThermoFisher) and TNFA levels (#BMS223HS, ThermoFisher); meanwhile, the tissue pellet was also collected and processed to check H3K9me2 (#P-3032, Epigentek) and H3K27me3 (#P-3020,Epigentek) levels by ELISA assay.

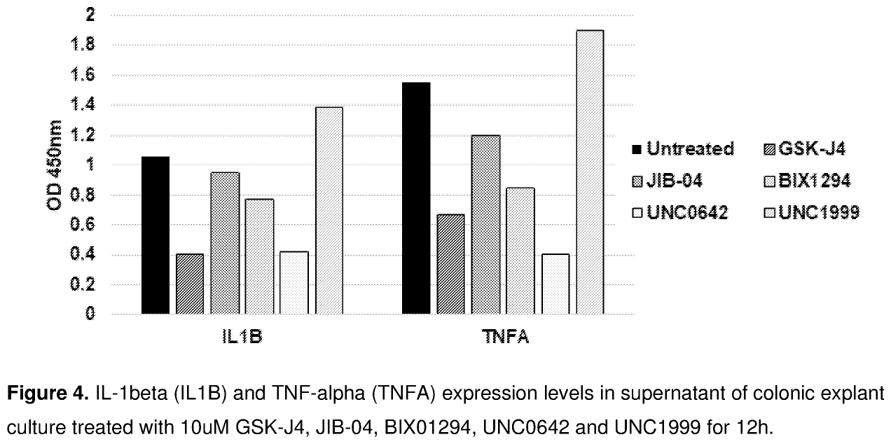

As the data shown in FIG. 3, colonic biopsy replicates treated with UNC0642, BIX01294, GSK-J4 and JIB-04 each had reduced IL-1B and TNFA levels, but colonic biopsy replicates treated with UNC1999 had increased IL-1B and TNFA levels (FIG. 4). Furthermore, the data shows that UNC0642 (targeting G9A) and GSK-J4 (targeting JMJD3) were more effective than BIX01294 and JIB-04 at reducing IL1B and TNFA levels.

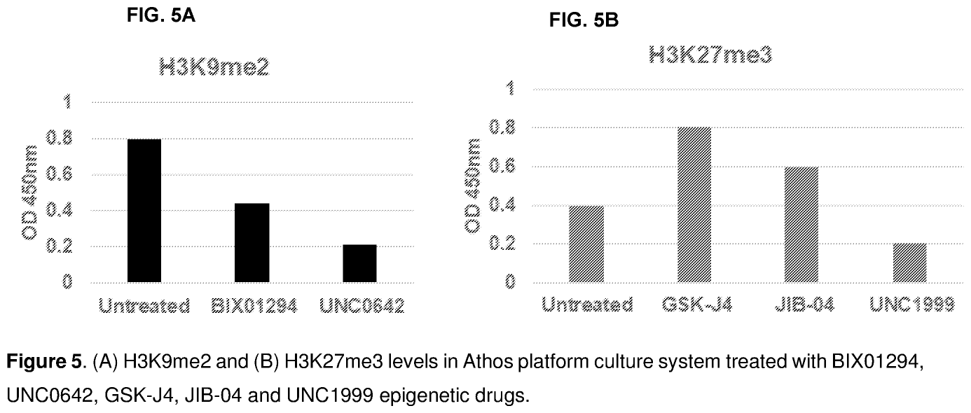

In addition, the feasibility of measuring chromatin alterations in colonic explant cultures and their potential use as biomarkers of response for chromatin modifying drugs were examined for the first time here. It was found that the G9A inhibitors BIX01294 and UNC0642 reduced the H3K9me2 levels, the JMJD3 inhibitors GSK-J4 and JIB-04 increased H3K27me3 levels, and the EZH2 inhibitor UNC1999 reduced H3K27me3 levels (FIG. 5). Currently, the state of art is to measure the expression levels of cytokines, such as IL1B and TNFA, in order to evaluate the efficacy of FDA-approved and new drugs for IBD patients. Here, however, it has been shown that alterations of specific histone modifications, such as H3K9me2 and H3K27me3, directly correlate to these cytokine level changes. Thus, instead of or in addition to measuring cytokine levels, histone modification levels can also be measured, as they can be predictive of a drug suppressing inflammation. This observation suggests for the first time that measuring histone-omic alterations in colonic explants from IBD patients could be a predictive surrogate biomarker of therapeutic response. In several embodiments, histone-omic alterations are measured in combination with another endpoint, for example cytokine levels.

Furthermore, the data suggests that G9A and JMJD3 inhibitors have anti-inflammatory activity and could be used as therapeutics for patients with IBD. Since compounds, non-limiting examples of which were used herein, regulate the levels of IL1B and TNFA, which are implicated in other autoimmune diseases, according to several embodiments such inhibitory compounds can be used in chronic inflammatory diseases, such as ulcerative colitis, Crohn's Disease, lupus, psoriasis and type I diabetes. On the other hand, EZH2 inhibitors exacerbated the inflammatory response, suggesting not only that they would not be effective as IBD therapeutics but also potentially could worsen the disease. This finding is important for additional reasons, since EZH2 inhibitors are used as cancer therapeutics (Italiano A et al., Lancet Oncol, 2018). These results suggest that monitoring GI symptoms in cancer patients treated with these drugs would be warranted.

### Example 3

This example describes the use of non-limiting embodiments of the ex vivo biopsy high throughput platform for identifying Transcriptome-Immunome multi-omic signature of response for antisense RNA inhibitors.

microRNAs are small RNA molecules that have been implicated in IBD pathogenesis, and inhibition of their expression could have therapeutic potential (de Souza H S P et al., Nat Rev Gastroenterol Hepatol, 2017). For example, it has been shown that miR-24-3p expression is up-regulated in UC colonic biopsies relative to controls, contributing to IBD pathogenesis (Wu F et al., Gastroenterology, 2008). Here, two non-limiting and novel antisense-miR-24-3p compounds were developed and their effects on cytokine production levels and miR-24 expression levels in UC active colonic biopsies were evaluated.

### Design of Antisense miR-24-3p Compounds

Sequence 1 (16 nt):
5-CTGCTGAACTGAGCCA-3
(seed sequence in bold)
miR-24-A compound:
(SEQ ID NO: 3)
5-CTG^{m}CTGAA^{m}CTGAG^{m}CCA-3
miR-24-B compound:
(SEQ ID NO: 4)
5-CTGCTGAACTGAGCCA-3

Chemical Modifications: Italics: locked nucleic acid (LNA) modification; m: methyl group in C; and underlined: phosphorothioate linkages. In other words, in miR-24-A, bases 1-16 comprise phosphorothioate linkages, bases 1-3, 10-11, and 15-16 comprise locked nucleic acids, and the cytosine at position 4, 9, and 14 comprises a methyl group (e.g., 5-methylcytosine). In miR-24-B, bases 1-16 comprise phosphorothioate linkages and bases 1-2, and 14-16 comprise locked nucleic acids.

The biopsy was processed as described in Example 1. Briefly, one well was used as control and treated with RNA-negative control (miR-NC, cat no. YI00199006, Qiagen) (1 uM), a second well was treated with the miR-24-A inhibitor (1 uM), and a third well was treated with miR-24-B inhibitor (1 uM). 50 uL of supernatant was collected to evaluate IL1B and TNFA levels and tissue pellet was collected and processed for RNA extraction (miRNeasy Kit, Qiagen). MicroRNA LNA miRNA PCR assays (Qiagen) were performed for miR-24-3p (cat.no YP00204260, Qiagen).

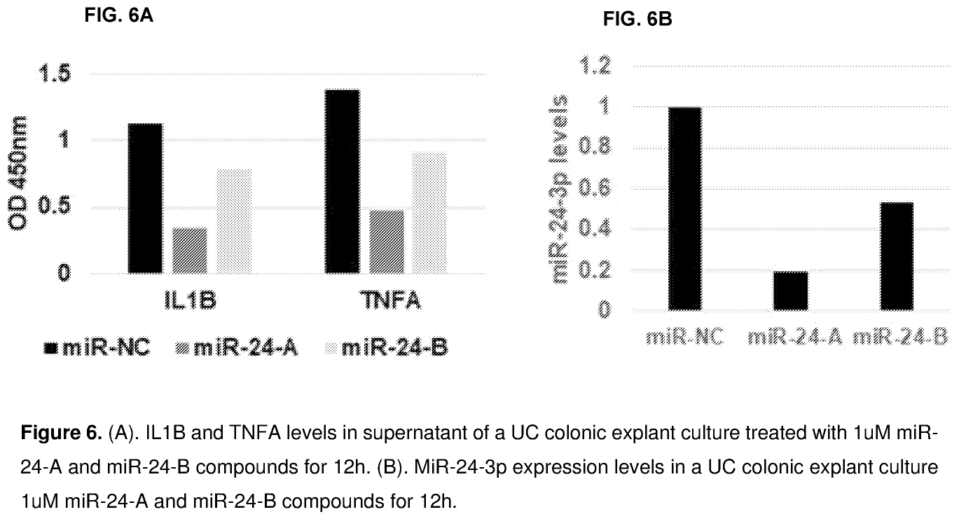

The data shows that MiR-24-A inhibitor was more effective in reducing IL1B and TNFA levels compared to miR-24-B inhibitor (FIG. 6A). Importantly, miR-24-A inhibitor more effectively suppressed miR-24-3p expression levels compared to miR-24-B inhibitor (FIG. 6B). It has been shown that miR-24-3p expression is up-regulated in UC colonic biopsies relative to controls, contributing to IBD pathogenesis (Wu F et al., Gastroenterology, 2008). Thus, these findings suggest that, according to some embodiments, examining the cytokine production levels in colonic explant cultures treated with RNA-targeting drugs should be performed in connection with RNA target gene levels and correlation with the cytokine response or other “omic” endpoint. Overall, the data show the feasibility of evaluating novel antisense compounds in the ex vivo biopsy high throughput platform.

Taken together, the ex vivo biopsy high throughput platform is a novel platform able to evaluate multi-omic signatures that predict drug responses in ˜24 hours. High throughput chemical compound screening in the ex vivo biopsy high throughput platform could rapidly identify the lead compounds that have the potential to be developed as IBD therapeutics and could be further tested in animal studies.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.”

Any titles or subheadings used herein are for organization purposes and should not be used to limit the scope of embodiments disclosed herein. Any terms defined herein shall also be given their ordinary meaning understood in the art.

1. An ex vivo method of screening one or more candidate therapeutics for treatment of a gastrointestinal disease, comprising:
receiving a biopsy sample from a patient with at least one active disease site,
separating the biopsy sample into a plurality of replicates, and
contacting a plurality of the replicates with an artificial tissue culture medium;
adding, to one or more of the individual replicates, a candidate therapeutic,
wherein at least one of the plurality of replicates is maintained as a control by contacting said replicate with an artificial tissue culture medium but not a candidate therapeutic;
culturing the plurality of placed replicates for a period of time; and
measuring, after the period of time, levels of one or more biomarkers from a replicate cultured with a candidate therapeutic and from a replicate cultured without a candidate therapeutic,
wherein the one or more biomarkers comprise at least one biomarker indicative of a histone modification; and
wherein a candidate therapeutic is identified when the candidate therapeutic induced changes in the at least one biomarker indicative of a histone modification such that the measured biomarker from the replicate cultured with the candidate therapeutic is significantly different from the measured biomarker from the replicate cultured without the candidate therapeutic.

2. The ex vivo method of claim 1, wherein the gastrointestinal disease is selected from inflammatory bowel disease, celiac disease, irritable bowel syndrome, and esophagitis.

3. The ex vivo method of claim 1, wherein the at least one biomarker indicative of a histone modification is measured by ELISA.

4. The ex vivo method of claim 1, wherein the at least one biomarker indicative of a histone modification measured is H3K9me2 or H3K27me3.

5. The ex vivo method of claim 1, wherein the at least one biomarker indicative of a histone modification measured is H3K9me2 and H3K27me3.

6. The ex vivo method of claim 1, wherein the at least one biomarker indicative of a histone modification measured is selected from H3K9me1/2/3, H3K27me1/2/3, H3K4me1/2/3, H3K36me1/2/3, H3K79me1/2/3, H3K9ac, H3K14ac, H3K18acm H3K56ac, H3ser10P, H3ser28P, total H3, H4K5ac, H4K8ac, H4K12ac, H4K16ac, H4R3m2a, H4R3m2s, H4K20me1/2/3, H4ser1, total H4, H3S10ph and H3S28ph, and/or the related histone-modifying genes: ASH1L, DOT1L, EHMT1, G9A/EHMT2, EZH1, EZH2, BMI1, SUZ12, MLL1-5, NSD1, PRDM2, SETD1-9, SETDB1/2, SETMAR, SMYD1-5, SUV39H1/2, SUV420H1/2, PRMT1-7, KDM1A/B, KMD2A/B, KMD3A/B, JMJDIC, KMD4A/B/C/D, KDM5A/B/C/D, KDM6A/B, GCN5, PCAF, HBO1,p300, CBP, SRC-1, ACTR, TIF2, TAF1, TFIIIC, HDAC1-11, and SIRT1-7.

7. The ex vivo method of claim 1, further comprising measuring the level of at least one cytokine associated with a gastrointestinal disease.

8. The ex vivo method of claim 7, wherein the at least one cytokine level measured is that of tumor necrosis factor alpha (TNFA) or interleukin 1B (IL1B).

9. The ex vivo method of claim 7, wherein the at least one cytokine level measured is that of TNFA and IL1B.

10. The ex vivo method of claim 7, wherein the at least one cytokine level measured is that of TNFA, IL1B, IL1RA, IL2, IL4, IL5, IL6, IL7, IL8, IL10, IL12, IL13, IL15, IL17A, IL17E, IL33, GM-CSF, G-CSF, ICAM1, MCP-1, IFNgamma. MIP1A, MIP1B, CCL5/RANTES, CCL11, CD40, CX3CL1, CXCL1, CXCL2, CXCL10, TREM1, MIP3A, and/or MIP3B.

11. The ex vivo method of claim 7, wherein the at least one cytokine level is measured by ELISA.

12. The ex vivo method of claim 11, wherein the TNFA, IL1B, H3K9me2, and H3K27me3 are measured by ELISA.

13. The method of claim 1, wherein the artificial tissue culture medium used to contact the plurality of replicates comprises L-alanyl-L-glutamine dipeptide.

14. The method of claim 13, wherein the concentration of L-alanyl-L-glutamine dipeptide artificial tissue culture medium is between 1 mM to 20 mM.

15. The method of claim 13, wherein the concentration of L-alanyl-L-glutamine dipeptide is between 2 mM and 6 mM.

16. (canceled)

17. (canceled)

18. (canceled)

19. The method of claim 1, further comprising measuring the expression levels of at least one epitranscriptomic regulator selected from the group consisting of a methyltransferase, a demethylase, a reader, and a deaminase.

20. The method of claim 19, wherein the methyltransferase is selected from METTL3, METTL14, METTL1, METTL6, METTL13, METTL16, NSUN2, NSUN3, NSUN4, TRMT2A, TRMT6, TRMT11, TRMT12, TRMT16B, BCDIN3D, KIAA1456, HENMT1, FBL, DIMT1, and combinations thereof.

21. (canceled)

22. (canceled)

23. (canceled)

24. A method according to any one of the preceding Claims, wherein the biopsy sample is obtained by colonoscopy, sigmoidoscopy, surgery, endoscopy, or gastroscopy.

25. (canceled)

26. (canceled)

27. The method of claim 24, wherein the biopsy sample is from a patient who is newly diagnosed with Ulcerative colitis, Crohn's Disease, Irritable Bowel Syndrome, Esophagitis, Celiac Disease, or has been treated with steroids, immunomodulators, and/or biologics.

28. (canceled)

29. (canceled)

30. (canceled)

31. (canceled)

32. (canceled)

33. (canceled)

34. (canceled)

35. (canceled)

36 (canceled)

37. (canceled)

38. (canceled)

39. (canceled)

40. (canceled)

41. (canceled)

42. (canceled)

43. (canceled)

44. (canceled)

45. (canceled)

46. A candidate therapeutic for the treatment of a gastrointestinal disease, wherein the candidate therapeutic was selected for use in the treatment by a method comprising:
culturing a plurality of replicates of a biopsy sample from a patient with at least one active gastrointestinal disease in an artificial tissue culture medium,
wherein each replicate is placed in its own individual tissue culturing environment;
adding, to one or more of the individual tissue culturing environments, a candidate therapeutic,
wherein at least one of the plurality of replicates is maintained as a control and is placed in an individual tissue culturing environment comprising an artificial tissue culture medium but not a candidate therapeutic;
culturing the plurality of placed replicates for a period of time; and
measuring, after the period of time, levels of one or more biomarkers from a replicate cultured with a candidate therapeutic and from a replicate cultured without a candidate therapeutic,
wherein a candidate therapeutic is identified when the candidate therapeutic induced changes in the one or more biomarkers such that the measured one or more biomarkers from the replicate cultured with the candidate therapeutic is significantly different from the measured one or more biomarkers from the replicate cultured without the candidate therapeutic.

47. The candidate therapeutic of claim 46, wherein the candidate therapeutic comprises:
a histone modifying factor selected from the group consisting of ASH1L, DOT1L, EHMT1, G9A/EHMT2,EZH1, EZH2, BMI1, SUZ12, MLL1-5, NSD1, PRDM2, SETD1-9, SETDB1/2, SETMAR, SMYD1-5, SUV39H1/2, SUV420H1/2, PRMT1-7, KDM1A/B, KMD2A/B, KMD3A/B, JMJDIC, KMD4A/B/C/D, KDM5A/B/C/D, KDM6A/B, GCN5, PCAF, HBO1,p300, CBP, SRC-1, ACTR, TIF2, TAF1, TFIIIC, HDAC1-11, SIRT1-7, and combinations thereof,
a chemical compound targeting a cytokine or chemokine selected from the group consisting of TNFA, IL1B, IL1RA, IL2, IL4, IL5, IL6, IL7, IL8, IL10, IL12, IL13, IL15, IL17A, IL17E, IL33, GM-CSF, G-CSF, ICAM1, MCP-1, IFNgamma. MIP1A, MIP1B, CCL5/RANTES, CCL11, CD40, CX3CL1, CXCL1, CXCL2, CXCL10, TREM1, MIP3A, MIP3B, and combinations thereof,
a chemical compound targeting an epitranscriptomic regulator, wherein the epitranscriptomic regulator is a methyltransferase,
wherein the methyltransferase is selected from the group consisting of METTL3, METTL14, METTL1, METTL6, METTL13, METTL16, NSUN2, NSUN3, NSUN4, TRMT2A, TRMT6, TRMT11, TRMT12, TRMT16B, BCDIN3D, KIAA1456, HENMT1, FBL, DIMT1, and combinations thereof
a chemical compound targeting an epitranscriptomic regulator, wherein the epitranscriptomic regulator is a demethylase,
wherein the demethylase is selected from the group consisting of ALKBH3, ALKBH5, FTO, and combinations thereof,
a chemical compound targeting an epitranscriptomic regulator, wherein the epitranscriptomic regulator is a reader,
wherein the reader comprises one or both of YTHDC2 and YTHDF2,
a chemical compound targeting an epitranscriptomic regulator, wherein the epitranscriptomic regulator is a deaminase,
wherein the deaminase comprises one or both of ADAR1 and ADAR2, and/or
an miR-24-3p inhibitor,
wherein the miR-24-3p inhibitor is an antisense-miR-24-3p compound, and
wherein the antisense-miR-24-3p compound has the sequence of SEQ ID NO: 1 (5-CTG^{m}CTGAA^{m}CTGAG^{m}CCA-3) or SEQ ID NO: 2 (5-CTGCTGAACTGAGCCA-3).

48. (canceled)

49. (canceled)

50. (canceled)

51. (canceled)

52. (canceled)

53. (canceled)


SEQUENCE LISTING (4 sequences)

SEQ ID NO: 1 - DNA, 16 bp, Artificial Sequence
  synthesized
  misc_feature (4)..(4): 5-methylcytosine
  misc_feature (9)..(9): 5-methylcytosine
  misc_feature (14)..(14): 5-methylcytosine
ctgctgaactgagcca

SEQ ID NO: 2 - DNA, 16 bp, Artificial Sequence
  synthesized
ctgctgaactgagcca

SEQ ID NO: 3 - DNA, 16 bp, Artificial Sequence
  synthesized
  misc_feature: miR-24-A
  misc (1)..(16): phosphorothioate linkages
  misc (1)..(3): Locked Nucleic Acids
  misc (4)..(4): 5-methylcytosine
  misc (9)..(9): 5-methylcytosine
  misc (10)..(11): Locked Nucleic Acids
  misc (14)..(14): 5-methylcytosine
  misc (15)..(16): Locked Nucleic Acids
ctgctgaactgagcca

SEQ ID NO: 4 - DNA, 16 bp, Artificial Sequence
  synthesized
  misc_feature: miR-24-B
  misc_feature (1)..(16): phosphorothioate linkages
  misc_feature (1)..(2): Locked Nucleic Acid
  misc_feature (14)..(16): Locked Nucleic Acid
ctgctgaactgagcca